(19) United States
(12) Patent Application Publication
(10) Pub. No.: US 2010/0305630 A1
(43) Pub. Date: Dec. 2, 2010

(54) HOUSING

(76) Inventors: Marcus Warwick, Cambridge (GB); Stuart Green, Lancashire (GB); David Hawks, Woodbury, MN (US)

Correspondence Address: HOFFMAN WARNICK LLC, 75 STATE STREET, 14TH FLOOR, ALBANY, NY, 12207, US

(21) Appl. No.: 12/744,365

(22) PCT Filed: Nov. 26, 2008
(86) PCT No.: PCT/GB08/51121

§ 371 Date: Aug. 12, 2010

(30) Foreign Application Priority Data
Nov. 27, 2007 (GB) 0723169.9

(51) Int. Cl. A61N 1/375; B29C 65/14

(52) U.S. Cl. 607/2; 156/272.2

(57) ABSTRACT

A first housing 2 includes a first part 4 and a second part 6, both of which comprise PEEK-OPTIMA (Trade Mark) polymer. The two parts 4, 6 are arranged to cooperate so that they can be laser welded and define a hermetically sealed housing 2 which may house electronic (and/or other) components and maintain them in a dry, sealed environment. The housing and contained electronic components may define an implantable device, for example a heart pacemaker, for long term implantation in a human body.

This invention relates to a housing and particularly, although not exclusively, relates to a housing for an implantable device for implantation in a human body and a method of making such a housing.

It is well known to provide implantable devices for implantation in a human body for applying a stimulus to a part of the body, for example a tissue, for a therapeutic purpose. Such implantable devices may be arranged to supply an electrical stimulus used in neurological therapy for stimulating nerves or muscle to combat pain or may be used as a heart pacemaker. Other applications include use in treating urinary urge incontinence by stimulating nerves close to the pelvic floor; and use in reduction of pressure sores by stimulating cavernous nerves. In addition, implantable devices are, in some cases, used to provide a chemical or mechanical stimulus.

Implantable devices may, in general terms, comprise a housing which includes electronic circuitry and a power source. Preferably, the housing defines an hermetically sealed environment so that its contents are protected from ingress of water which could be damaging. It has been known for over 30 years to make such housing out of titanium parts which are welded together to define a hermetically sealed container. In addition, housings made from plastics materials have been proposed but have generally been inadequate due to difficulties in forming a hermetic seal between two plastic components used to define the housing.

US2006/0259090 describes implantable medical devices with polymer-polymer interfaces. To join first and second plastic components of the device to define a housing, a light absorbing metal layer is disposed on a flange or rim at an interface between the plastics components. The metal layer may be formed by physical or vapour chemical deposition, sputter coating, electroplating or electroless plating. The interface is then heated and the metal layer absorbs heat which facilitates the bonding of the flanges/rims of the first and seconds plastics components to one another.

It is an object of the present invention to address problems with housings made from plastics materials.

It is another object of the present invention to address problems associated with joining first and second plastics components to define a substantially hermetic seal between the components.

According to a first aspect of the invention, there is provided a method of joining a first part to a second part, wherein the first part includes a first interface which is arranged to be joined to a second interface defined by the second part, wherein the first interface of the first part includes a first side and a second side, the method comprising:

directing radiation from a first position which is closer to the first side of the first interface to a position which is closer to the second side of the first interface, the radiation being directed to heat predominantly an area of the first interface of the first part which is closer to the second side of the first interface than it is to the first side of the first interface.

Said radiation may be supplied from a radiation device. Said radiation device is preferably arranged to heat a said area of the first interface. Said radiation device may be a source of heat, for example focussed infrared radiation. Said radiation device is preferably a laser. The radiation device may emit radiation at least one wavelength in the range 900-1000 nm, preferably in the range 920-960 nm. The radiation device may have an energy in the range 10 W-200 W, preferably 20 W to 100 W. Said radiation device may be arranged to direct radiation to a relatively small area of the first part. For example, said small area may have a maximum diameter of less than 1 cm, preferably less than 0.7 cm, more preferably less than 0.5 cm. Said area may be in the range 0.03 cm² to 3 cm², preferably in the range 0.1 cm² to 0.5 cm². Said radiation device is preferably arranged to be focussed on said relatively small area.

Said first interface preferably defines a frusto-conical surface.

The thickness of the first part underlying said first interface and measured in a direction which is transverse to, for example perpendicular to, the first interface is preferably smaller in a region which underlies said first interface at a position which is closer to the second side of the first interface than it is to said first side.

The thickness of the first part underlying said first interface and measured as aforesaid preferably increases (preferably gradually and preferably at a substantially constant rate) on moving from a position which is closer to (and preferably is adjacent to) the second side of the first interface to a position which is closer to (and preferably is adjacent to) the first side of the first interface.

Said first part preferably tapers inwardly on moving from its first side to its second side.

Said first part preferably defines a pointed region at its second side.

Said first part preferably includes a first outwardly facing surface. An angle defined between the first interface and said first outwardly facing surface is preferably in the range 200° to 250°, more preferably 210° to 240° and especially is about 225°. Said first outwardly facing surface and said first interface suitably intersect. Said first outwardly facing surface is preferably cylindrical.

Said first part preferably includes an inwardly facing surface. An angle defined between the first interface and said inwardly facing surface is preferably in the range 200° to 250°, preferably 210° to 240° and especially about 225°. Said inwardly facing surface is preferably cylindrical.

Said first outwardly facing surface and said inwardly facing surface preferably extend substantially parallel to one another. Said surfaces are preferably co-axial.

Said first part preferably includes said first outwardly facing surface and said inwardly facing surface, with said first interface extending between and suitably being contiguous with said surfaces.

Said first part preferably include a first guide surface which is arranged to guide movement of the second part as the first and second parts are engaged prior to being joined. Said first guide surface preferably faces outwardly. Said first guide surface is preferably cylindrical and preferably has a circular cross-section. It suitably extends substantially parallel to said first outwardly facing surface of said first part. A first gap is preferably defined between said first guide surface and said inwardly facing surface of said first part. Said first guide surface is preferably cylindrical.

Said first interface is preferably annular. It preferably has a substantially circular cross-section.

Said first interface may have a width which is less than one tenth, preferably less than one-twentieth, of its length (e.g. its circumference when the interface is annular). Said first interface may have a width of at least 1 mm, preferably at least 1.5 mm. The width may be less than 10 mm, preferably less than 5 mm.

Said first part is preferably a component of a housing which comprises said first part and said second part. The housing is preferably hollow. It may be substantially cylindrical. Said housing may be arranged to house electronic components for example a medical device for implantation in a human body.

Said second interface preferably defines a frusto-conical surface.

Said second part preferably includes a second outwardly facing surface. An angle defined between the second interface and said second outwardly facing surface is preferably in the range 200° to 250°, more preferably 210° to 240° and especially is about 225°. Said second outwardly facing surface and said second interface suitably intersect. Said outwardly facing surface is preferably cylindrical.

Said second part preferably includes an inwardly facing surface. An angle defined between the second interface and said inwardly facing surface is preferably in the range 200° to 250°, preferably 210° to 240° and especially is about 225°. Said inwardly facing surface is preferably cylindrical.

Said second outwardly facing surface and said inwardly facing surface of said second part preferably extend substantially parallel to one another. Said surfaces are preferably co-axial.

Said second part preferably includes said second outwardly facing surface and said inwardly facing surface, with said second interface extending between and suitably being contiguous with said surfaces.

Said second part preferably includes a second guide surface which is arranged to cooperate with the first part as (e.g. the first guide surface thereof) as the first and second parts are engaged prior to being joined. Said second guide surface preferably faces inwardly. Said second guide surface is preferably cylindrical and preferably has a circular cross-section. It suitably extends substantially parallel to said second outwardly facing surface of said second part. A second gap is preferably defined between said second inwardly facing surface of said second part and said first guide surface of said first part.

When respective first and second gaps are defined by said first and second parts, the gaps are preferably aligned to define a combined gap. Said combined gap may be arranged to accommodate elements of said first and second interface regions after the first and second parts have been joined in the method.

Said second interface is preferably annular. It preferably has a substantially circular cross-section.

In the method, the first and second parts are suitably selected and engaged so that the first and second interfaces face one another and preferably a region of the first interface abuts a region of the second interface. Preferably, initially, a region of the first interface at or adjacent the second side thereof abuts a region of the second interface at or adjacent the second side thereof. Suitably, therefore, a narrow region of said first interface abuts the second interface.

Suitably, an angle in the range 65° to 115°, preferably 80 to 100°, more preferably 85 to 95°, especially about 90° is defined between the first and second interfaces prior to being joined. The interfaces may together define a groove, which is suitably annular and is suitably outwardly facing. Said radiation may in the method be directed into said groove, suitably predominantly towards a bottom or closed end thereof. The groove may have a substantially V-shaped cross-section.

The method preferably comprises causing the first and second parts to move relative to one another. To this end, one of the parts may not move translationally and the other part may move translationally. A force in the range 50-500N, preferably in the range 100-250N may be applied to urge the parts towards one another. With radiation being directed as described, the first interface may be softened so that it may turn towards the second interface and make face to face contact therewith.

In a preferred embodiment, the second interface may also be softened by said radiation. The second interface may turn towards the first interface so the first and second interfaces may make face to face contact.

During movement of said interfaces, respective guide surfaces (which are preferably cylindrical as described above) of said first and second parts may slide over one another.

In the method heat is suitably applied to predominantly heat an area of the first interface which is closer to the second side of the first interface. Preferably, heat is applied, suitably from said first position (and suitably from the same source of radiation which applies heat to the first interface) to heat predominantly an area of the second interface which is closer to a second side of the second interface, wherein said first side of the second interface is closer to said first position than said second side of said second interface is to said first position.

In a preferred embodiment wherein the first and second interfaces define a groove, radiation is directed from said first position towards a bottom of the groove which bottom is at the respective second sides of the first and second interfaces, to predominantly heat areas of the interfaces at the bottom of the groove.

The method preferably comprises causing the first and second parts to rotate, suitably so that radiation may be directed around respective annular parts of the first and second parts being joined together. Suitably, said first and second parts are rotational fixed relative to each other.

Radiation is suitably directed from said first position which remains at a fixed position during the time it is directing radiation towards said first and/or second parts.

In some embodiments, radiation may be directed from a plurality, for example 4 or more positions, which may be positioned at intervals around said first interface.

The method may involve directing radiation at said first and second parts for a time of less than 60 seconds, or less than 30 seconds. It will be appreciated that the method suitably involves laser welding.

Said first part and/or said second part may comprise a plastic material which may comprise a bio-compatible polymeric material.

Said bio-compatible polymeric material may be any polymeric material which is non-toxic and not otherwise harmful when introduced into the human body.

Said plastic material, for example said bio-compatible polymeric material may have a Notched Izod Impact Strength (specimen 80 mm×10 mm×4 mm with a cut 0.25 mm notch (Type A), tested at 23° C., in accordance with ISO180) of at least 4 KJm⁻², preferably at least 5 KJm⁻², more preferably at least 6 KJm⁻². Said Notched Izod Impact Strength, measured as aforesaid, may be less than 10 KJm⁻², suitably less than 8 KJm⁻².

The Notched Izod Impact Strength, measured as aforesaid, of the plastic material may be at least 3 KJm⁻², suitably at least 4 KJm⁻², preferably at least 5 KJm⁻². Said impact strength may be less than 50 KJm⁻², suitably less than 30 KJm⁻².

Said plastic material suitably has a melt viscosity (MV) of at least 0.06 kNsm⁻², preferably has a MV of at least 0.09 kNsm⁻², more preferably at least 0.12 kNsm⁻², especially at least 0.15 kNsm⁻².

MV is suitably measured using capillary rheometry operating at 400° C. at a shear rate of 1000 s⁻¹ using a tungsten carbide die, 0.5×3.175 mm.

Said plastic material may have a MV of less than 1.00 kNsm⁻², preferably less than 0.5 kNsm².

Said plastic material may have a MV in the range 0.09 to 0.5 kNsm⁻², preferably in the range 0.14 to 0.5 kNsm⁻².

Said plastic material may have a tensile strength, measured in accordance with ISO527 (specimen type Ib) tested at 23° C. at a rate of 50 mm/minute of at least 20 MPa, preferably at least 60 MPa, more preferably at least 80 MPa. The tensile strength is preferably in the range 80-110 MPa, more preferably in the range 80-100 MPa.

Said plastic material may have a flexural strength, measured in accordance with ISO178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa.

Said plastic material may have a flexural modulus, measured in accordance with ISO178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

Said plastic material may be amorphous or semi-crystalline. It is preferably semi-crystalline.

The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning Calerimetry (DSC).

The level of crystallinity of said plastic material may be at least 1%, suitably at least 3%, preferably at least 5% and more preferably at least 10%. In especially preferred embodiments, the crystallinity may be greater than 25%.

The main peak of the melting endotherm (Tm) of said plastic material (if crystalline) may be at least 300° C.

Said plastic material may include a polymeric moiety which is: an acrylate (e.g. it comprises or consists of methylmethacrylate moieties); a urethane; a vinyl chloride; a silicone; a siloxane (eg comprising dimethylsiloxane moieties); a sulphone; a carbonate; a fluoroalkylene (e.g. a fluoroethylene); an acid (e.g. a glycolic acid or lactic acid); an amide (e.g. comprising nylon moieties); an alkylene (e.g. ethylene or propylene); an oxyalkylene (e.g. polyoxymethylene); an ester (e.g. polyethylene terephthalate), an ether (e.g. an aryletherketone, an arylethersulphone (e.g. polyethersulphone or polyphenylenesulphone) or an ether imide).

Said plastic material may be selected from a polyalkylacrylate (e.g. polymethylmethacrylate), a polyfluoroalkylene (e.g. PTFE), a polyurethane, a polyalkylene (e.g. polyethylene or polypropylene), a polyoxyakylene (e.g. polyoxymethylene), a polyester (e.g. polyethylene terephthalate or polybutylene terephthalate), a polysulphone, a polycarbonate, a polyacid (e.g. polyglycolic acid or polylactic acid), a polyalkylene oxide ester (e.g. polyethylene oxide terephalate) a polyvinylchloride, a silicone, a polysiloxane, a nylon, a polyaryletherketone, a polarylethersulphone, a polyether imide and any copolymer which includes any of the aforementioned.

Preferably, said plastic material is selected from polyethylene, polypropylene, silicone and polyetheretherketone. More preferably, said polymeric material is selected from polyethylene, polypropylene, silicone and polyetheretheketone.

Said plastic material may include a repeat unit of general formula

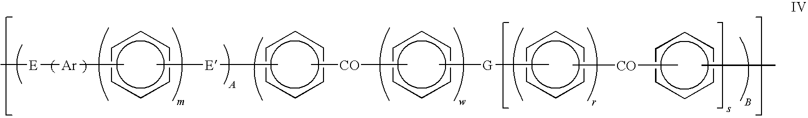

or a repeat unit of general formula

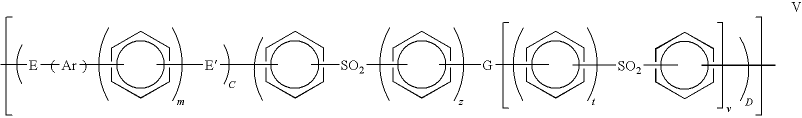

wherein A, B, C and D independently represent 0 or 1,
E and E′ independently represent an oxygen or a sulphur atom or a direct link, G represents an oxygen or sulphur atom, a direct link or a —O-Ph-O— moiety where Ph represents a phenyl group, m, r, s, t, v, w, and z represent zero or 1 and Ar is selected from one of the following moieties (i) to (v) which is bonded via one or more of its phenyl moieties to adjacent moieties

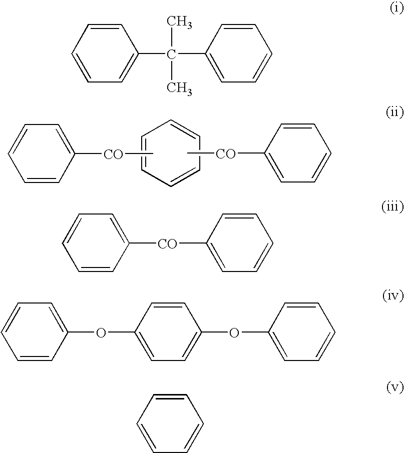

Unless otherwise stated in this specification, a phenyl moiety has 1,4-, linkages to moieties to which it is bonded.

Said plastic material may be a homopolymer which includes a repeat unit of IV or V or may be a random or block copolymer of at least two different units of IV and/or V.

As an alternative to a plastic material comprising units IV and/or V discussed above, said plastic material may include a repeat unit of general formula

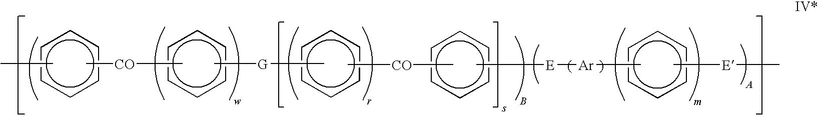

or a homopolymer having a repeat unit of general formula

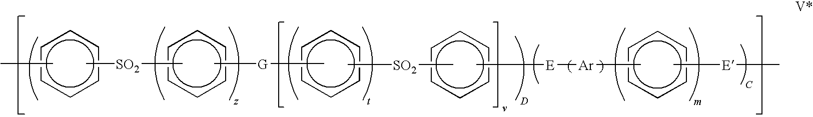

wherein A, B, C, and D independently represent 0 or 1 and E, E′, G, Ar, m, r, s, t, v, w and z are as described in any statement herein.

Said plastic material may be a homopolymer which includes a repeat unit of IV* or V* or a random or block copolymer of at least two different units of IV* and/or V*.

Preferably, said plastic material is a homopolymer having a repeat unit of general formula IV.

Preferably Ar is selected from the following moieties (vi) to (x)

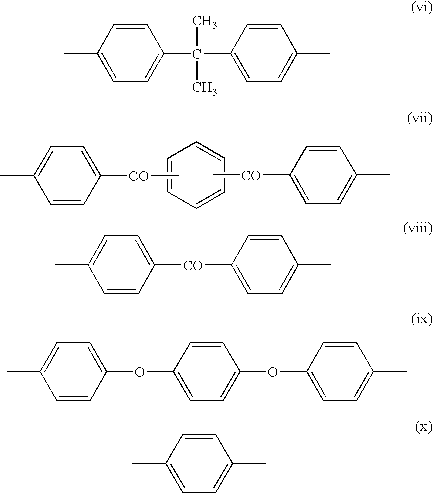

In (vii), the middle phenyl may be 1,4- or 1,3-substituted. It is preferably 1,4-substituted.

Suitable moieties Ar are moieties (ii), (iii), (iv) and (v) and, of these, moieties, (ii), (iii) and (v) are preferred. Other preferred moieties Ar are moieties (vii), (viii), (ix) and (x) and, of these, moieties (vii), (viii) and (x) are especially preferred.

An especially preferred class of plastic materials are polymers (or copolymers) which consist essentially of phenyl moieties in conjunction with ketone and/or ether moieties. That is, in the preferred class, the first polymer material does not include repeat units which include —S—, —SO₂— or aromatic groups other than phenyl. Preferred plastic materials, for example bio-compatible polymeric materials of the type described include:
(a) a polymer consisting essentially of units of formula IV wherein Ar represents moiety (v), E and E′ represent oxygen atoms, m represents 0, w represents 1, G represents a direct link, s represents 0, and A and B represent 1 (i.e. polyetheretherketone).
(b) a polymer consisting essentially of units of formula IV wherein E represents an oxygen atom, E′ represents a direct link, Ar represents a moiety of structure (ii), m represents 0, A represents 1, B represents 0 (i.e. polyetherketone);
(c) a polymer consisting essentially of units of formula IV wherein E represents an oxygen atom, Ar represents moiety (ii), m represents 0, E′ represents a direct link, A represents 1, B represents 0, (i.e. polyetherketoneketone).
(d) a polymer consisting essentially of units of formula IV wherein Ar represents moiety (ii), E and E′ represent oxygen atoms, G represents a direct link, m represents 0, w represents 1, r represents 0, s represents 1 and A and B represent 1. (i.e. polyetherketoneetherketoneketone).
(e) a polymer consisting essentially of units of formula IV, wherein Ar represents moiety (v), E and E′ represents oxygen atoms, G represents a direct link, m represents 0, w represents 0, s, r, A and B represent 1 (i.e. polyetheretherketoneketone).
(f) a polymer comprising units of formula IV, wherein Ar represents moiety (v), E and E′ represent oxygen atoms, m represents 1, w represents 1, A represents 1, B represents 1, r and s represent 0 and G represents a direct link (i.e. polyether-diphenyl-ether-phenyl-ketone-phenyl-).

Said plastic material may consist essentially of one of units (a) to (f) defined above. Alternatively, said plastic material may comprise a copolymer comprising at least two units selected from (a) to (f) defined above. Preferred copolymers include units (a). For example, a copolymer may comprise units (a) and (f); or may comprise units (a) and (e).

Said plastic, for example bio-compatible polymeric material preferably comprises, more preferably consists essentially of, a repeat unit of formula (XX)

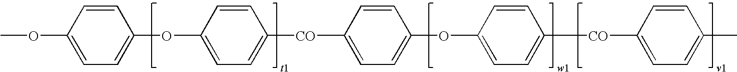

where t1, and w1 independently represent 0 or 1 and v1 represents 0, 1 or 2. Preferred plastic materials have a said repeat unit wherein t1=1, v1=0 and w1=0; t1=0, v1=0 and w1=0; t1=0, w1=1, v1=2; or t1=0, v1=1 and w1=0. More preferred have t1=1, v1=0 and w1=0; or t1=0, v1=0 and w1=0. The most preferred has t1=1, v1=0 and w1=0.

In preferred embodiments, said plastic material is selected from polyetheretherketone, polyetherketone, polyetherketoneetherketoneketone and polyetherketoneketone. In a more preferred embodiment, said plastic material is selected from polyetherketone and polyetheretherketone. In an especially preferred embodiment, said plastic material is polyetheretherketone.

Said first part preferably includes radiation absorbing means which is suitably arranged to absorb applied radiation and thereby allow said area of said first interface to be heated. Said first part may include at least 0.05 wt %, preferably at least 0.1 wt % of said radiation absorbing means. Said first part may include 0.5 wt % to 5 wt %, suitably 0.5 wt % to 3 wt %, of said radiation absorbing means. In one embodiment, said second part may also include radiation absorbing means. Any suitable type of radiation absorbing means may be used. Such means is suitably bio-compatible. It is preferably carbon-containing. Preferably, said radiation absorbing means comprises carbon black.

Said radiation absorbing means is preferably dispersed throughout a said bio-compatible polymeric material, suitably so it is homogenously dispersed.

Preferably, in the method the first and second parts which comprise plastic material (which may be the same or different for the respective parts) are contacted directly, with no solid material (preferably no other material at all) being arranged between the first and second parts.

Preferably, said first and second parts include the same bio-compatible polymeric material. They may differ in terms of whether or not a filler or fillers is/are dispersed in said polymeric material.

Said first and second parts are preferably arranged to be joined to define a housing suitable for implantation into a human body. The first and second parts may be arranged to be joined and define a substantially impermeable and/or sealed region at least between the first and second interfaces which are joined in the method. A hermetic seal may be defined between the first and second parts.

The method of the first aspect may include joining said first and second parts with an electronic device positioned between said first and second parts so that when the parts are joined said electronic device is positioned between said first and second parts, for example in a housing which is formed. The electronic device may be arranged to apply a stimulus to a part of the body in which it may be implanted in use. For example, said electronic device may comprise a heart pacemaker.

Said first part may have no dimension which is greater than 20 cm, preferably no dimension greater than 10 cm. Said second part may have no dimension which is greater than 20 cm, preferably no dimension greater than 10 cm.

According to a second aspect of the invention, there is provided a housing made in a method according to the first aspect.

According to a third aspect, there is provided a housing which comprises a first part and a second part which are joined to define the housing, wherein said first part includes a first guide surface which faces outwardly and abuts a second guide surface which faces inwardly wherein a gap (referred to in the first aspect as “a combined gap”) is defined outwardly of said first guide surface in which joined interfaces of the first and second parts are contained, at least in part. A hollow and/or free volume may be defined in said gap around said joined interfaces.

According to a fourth aspect of the invention, there is provided a first part and a second part, each being as described according to the first aspect, said first and second parts being arranged to be joined together by use of radiation.

Respective first and second interfaces of said first and second parts are preferably arranged to define an annular groove (e.g. of V-section), wherein radiation may be directed towards the bottom of the groove for joining the first and second parts together.

According to a fifth aspect of the invention, there is provided a housing which includes a first part joined to a second part, said first part comprising a plastic material and said second part comprising a plastic material.

Said housing preferably includes a combined gap (referred to in the first aspect) in which parts of the joined interface regions are contained. The combined gap includes a wall which is arranged to shield components arranged within the housing from contact by parts of the joined interface regions during joining of said interface regions.

According to a sixth aspect there is provided a first part per se for joining to a second part.

According to a seventh aspect, there is provided a second part per se for joining to a first part.

Any feature of any aspect of any invention or embodiment described herein may be combined with any feature of any aspect of any other invention or embodiment described herein mutatis mutandis.

Specific embodiments of the invention will now be described, by way of examples, with reference to the accompanying drawings, in which:

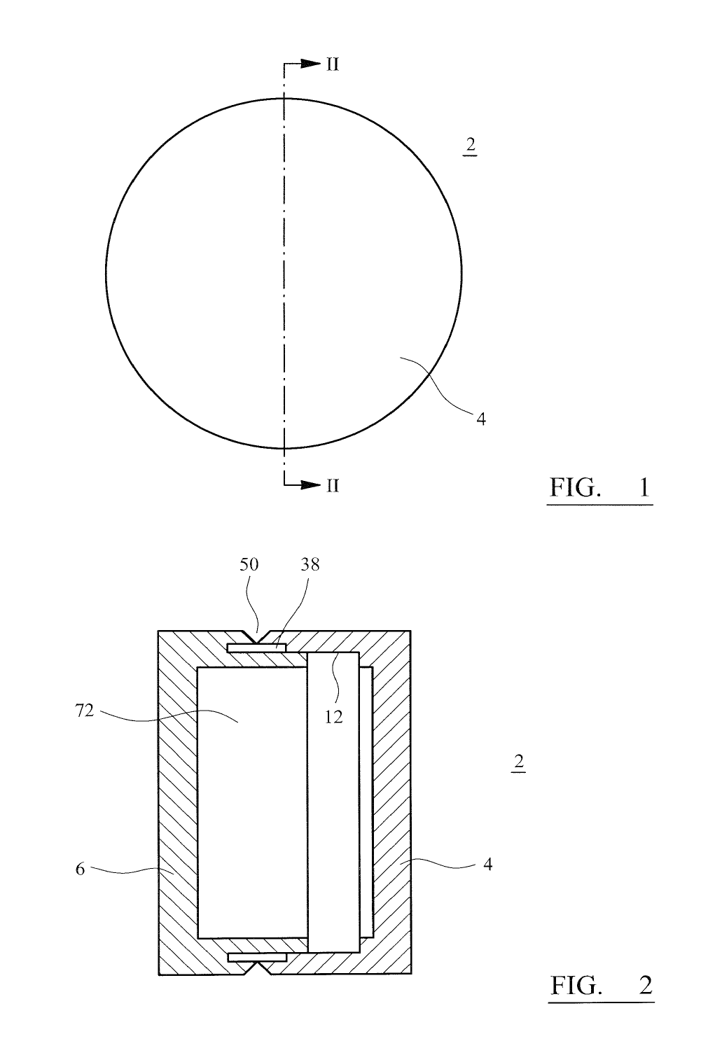

FIG. 1 is an end view of a first housing prior to welding;

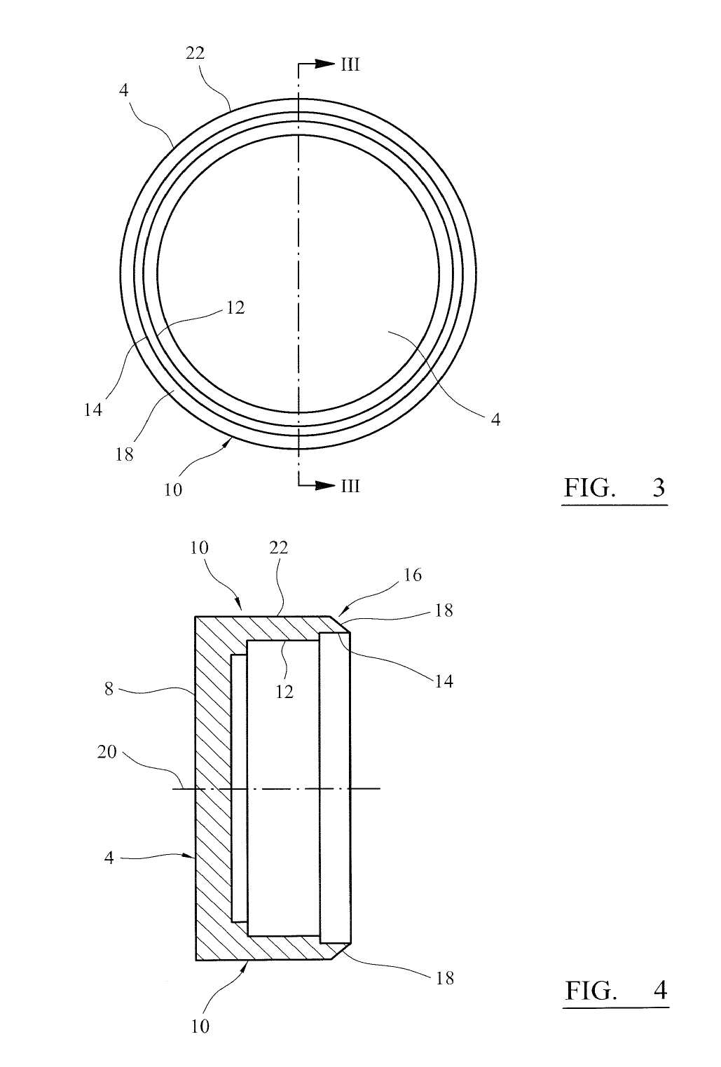

FIG. 2 is a cross-section along line II-II of FIG. 1;

FIG. 3 is an internal view of a first part of the first housing;

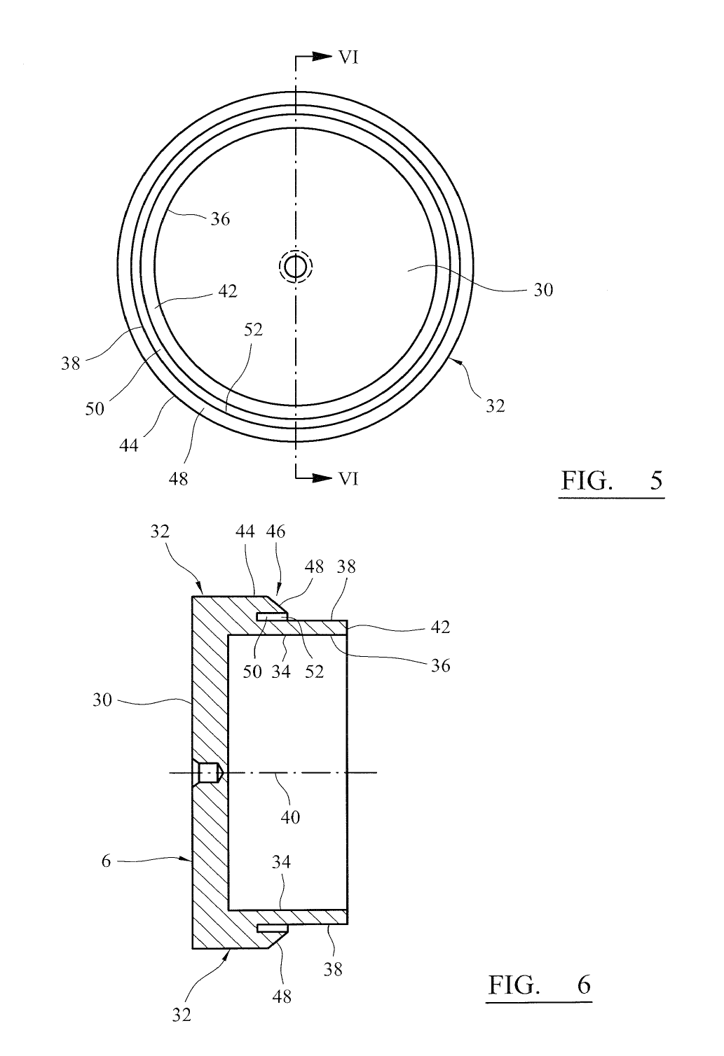

FIG. 4 is a cross-section along line III-III of FIG. 3;

FIG. 5 is an internal view of a second part of the first housing;

FIG. 6 is a cross-section along line VI-VI of FIG. 5;

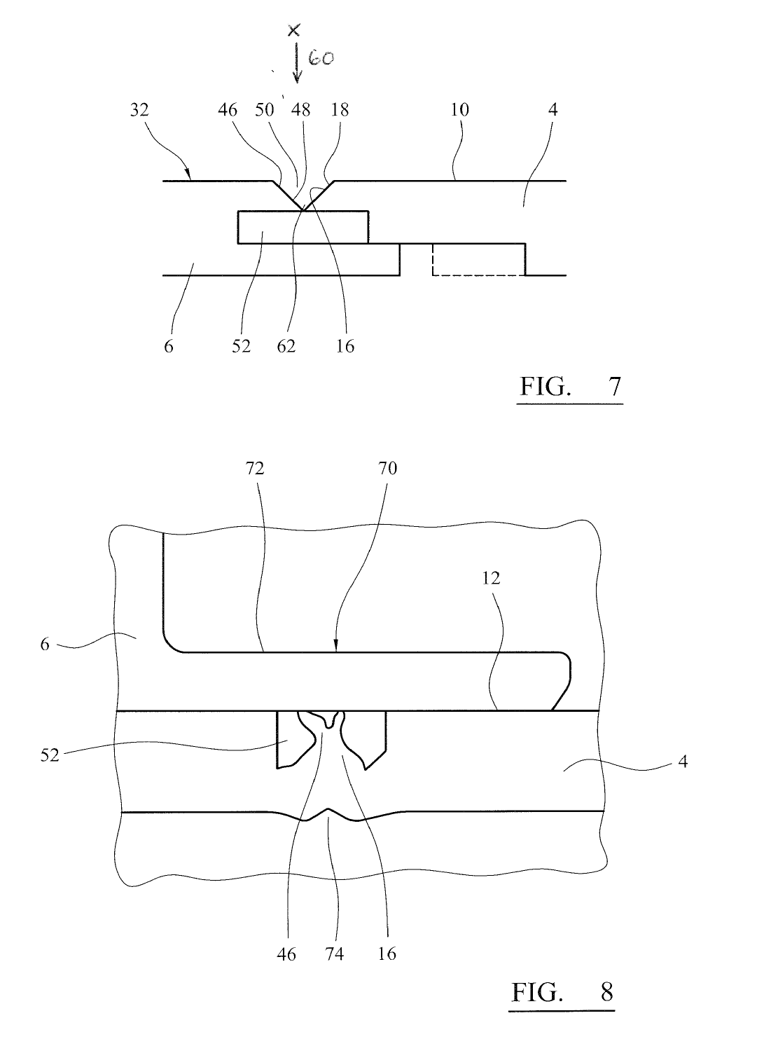

FIG. 7 is an enlarged view of joint portions of the first and second parts prior to welding the joint portions;

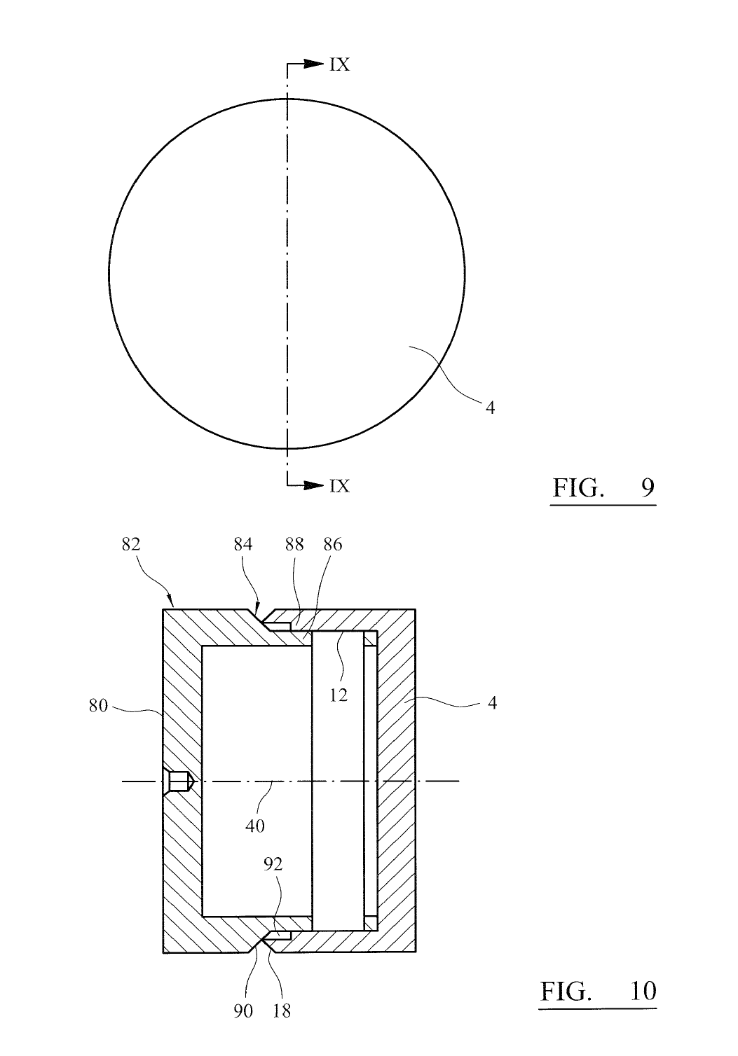

FIG. 8 is a photograph of a joint after welding the joint portions;

FIG. 9 is an end view of a second housing prior to welding;

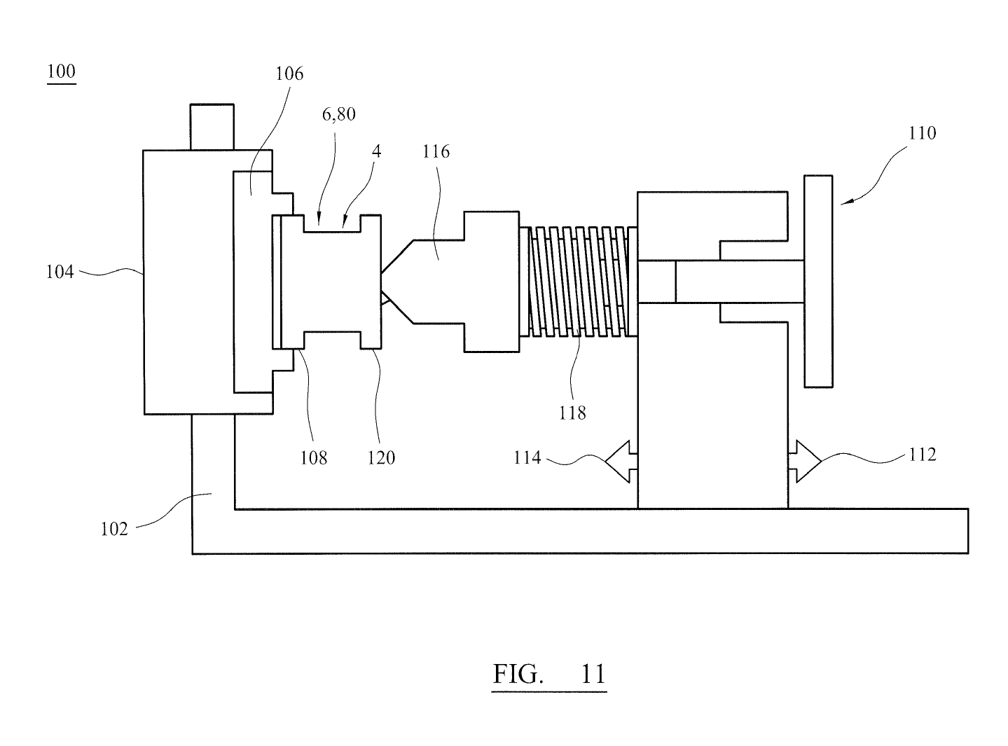

FIG. 10 is a cross-section along line IX-IX of FIG. 9; and

FIG. 11 is an schematic side view of a rotating clamping jig.

The following material is referred to hereinafter:

PEEK-OPTIMA (Trade Mark)—an implantable grade of polyetheretherketone, obtained from Invibio Limited, Thornton Cleveleys, UK.

A first housing 2 includes a first part 4 and a second part 6, both of which comprise PEEK-OPTIMA (Trade Mark) polymer. The two parts 4, 6 are arranged to cooperate so that they can be laser welded and define a hermetically sealed housing 2 which may house electronic (and/or other) components and maintain them in a dry, sealed environment. The housing and contained electronic components may define an implantable device, for example a heart pacemaker, for long term implantation in a human body. Further details on such an implantable device and housing therefor are provided below.

Referring to FIGS. 3 and 4, the first part 4 may be machined from stock or injection moulded from a material comprising PEEK-OPTIMA (Trade Mark) polymer by a conventional process. The part 4 includes an end region 8 and a circularly cylindrical wall 10 which is internally stepped to define a first circularly cylindrical guide surface 12 and a distal annular step 14. An outer annular edge of wall 10 defines triangular cross-section annular region 16 which includes an annular abutment surface 18 which extends inwardly at an angle of 45° relative to a central axis 20 about which wall 10 is defined. On its outside, wall 10 defines a smooth cylindrical surface 22.

Referring to FIGS. 5 and 6, the second part 6 is also machined from a material comprising PEEK-OPTIMA (Trade Mark) polymer by a conventional process. The part 6 includes an end region 30 and an annular wall 32. Wall 32 includes an inner annular member 34 which has a smooth inwardly facing annular surface 36 and a smooth outwardly facing annular surface 38 which extends parallel to axis and is centred on it. End surface 42 is defined between the annular surfaces 36, 38.

Wall 32 also includes an outer annular member 44 which has an outer annular edge which defines triangular cross-section annular region 46 which includes an annular abutment surface 48 which extends inwardly at an angle of 45° relative to axis 40.

The outer annular member 44 is shorter than the inner member 34 and an annular gap 50 is defined between outwardly facing surface 38 and inwardly facing surface 52 which extends parallel to axis 40 and is centred on it.

The first part 4 and second part 6 are co-operable as shown in FIGS. 2 and 7. In this regard, the first part 4 can be mounted so that its first cylindrical surface 12 slidably engages the annular surface 38 of the second part 6. When so disposed, the triangular cross-section regions 16, 46 are aligned so that the vertices of the triangular cross-sections abut and “V”-shaped band 50 is defined between first and second housing parts 4, 6. Additionally and most clearly seen in FIG. 7, an annular void 52 is defined between the parts 4, 6.

The parts, 4, 6 may be laser welded together by directing a laser, from a position X (FIG. 7) in the direction of arrow 60, at the apex of V-shaped band 50 so as to initially preferentially heat the bottom region 62 of the V. At the same time, the parts 4, 6 are spun about aligned axes 20, 40 so that the laser heats the parts 4, 6 around their circumference to substantially the same extent. Additionally, during this time, an axial force is applied between parts 4 and 6 to urge them towards one another.

As the parts 4, 6 are heated, spun and the axial force applied, the triangular regions 16, 46 soften and gradually collapse inwardly as abutment surfaces 18, 48 move closer together. The abutment surfaces 18, 48 contact one another initially at their inner edges and as parts 4, 6 move closer, their outer edges move closer together and eventually abut. As a result of the heating and abutment under force of the abutment surfaces 18, 48, the surfaces become welded together. It will be appreciated that, as the inner edges abut, the laser will gradually apply heat to parts of abutment surfaces 18, 48 which are disposed further and further outwardly, until just prior to abutment surfaces 18, 48 making complete face to face contact, the laser will be applying heat into a V-shaped region which is only slightly depressed below the outer surfaces of the walls 10 and 32.

It will be appreciated that, during laser welding of parts 4, 6, surfaces 12 and 38 slide over one another and the triangular regions 16, 46 collapse into annular void 52. This is shown in FIG. 8 from which it will be noted that void 52 contains the collapsed triangular regions 16, 46 and that a region 70 opposite the void 52 and positioned within the housing is not distorted. Advantageously, therefore, housing void 72 is not distorted and/or no elements of first and/or second parts 4, 6 enter it during laser welding. This may help to avoid damage of electronic and/or other parts which may be contained within housing void 72, during the laser welding process. It will also be noted from FIG. 8 that there is only limited weld flash 74 on the outside of the laser welded housing. It is believed it should be possible to substantially eliminate such flash by optimisation of the dimensions of parts 4, 6, the force applied between the two parts, the speed of rotation during laser welding, the energy supplied by the laser and the time of its application.

The parts 4, 6 are arranged to define a substantially symmetrical joint. However, asymmetrical joints may be defined using part 4 in conjunction with a part 80, as shown in FIG. 10. The part 80 includes a circular cylindrical wall 82 which includes a proximal inwardly extending, at 45°, annular region 84 and a distal region 86 having an outwardly facing annular surface 88 which surface 12 of part 4 is arranged to slidably engage. Annular region 84 defines an outwardly facing abutment surface 85. A generally V-shaped band 90 and a void 92 are also defined between parts 4, 80.

The parts 4, 80 may be laser welded generally as described above for parts 4, 6. In this case, abutment surface 18 of part 4 is softened and urged inwardly against abutment surface 85 of part 90 which will also be softened, so that surfaces 18 and 85 become welded.

FIG. 11 shows apparatus 100 which may be used in the laser welding of part 4 with part 6 or 80. The apparatus includes a rigid frame 102 which supports an electric motor 104 which is operatively connected to a chuck 106 having a holder 108 which is arranged to releasably secure a part 6 or 80 so that the part may be rotated about its axis. A support 110 is mounted on the rigid frame 102 and is movable linearly as represented by arrows 11, 114. The support 110 includes a rotating support 116 which is resiliently loaded by means of compression spring 118. The support 116 carries a holder 120 which in turn releasably secures part 4 in position. A laser (not shown) is arranged to direct a laser beam into a V-shaped band 50 defined between parts 4 and 6 or 80. The laser may be a Laserlines (Trade Mark) direct diode laser, operating at a wavelength of 940 nm and having a maximum power output of 150 W.

In addition, means is provided for subjecting irradiated surfaces of parts 4, 6, 80 with an inert (e.g. argon or nitrogen) gas jet to prevent the parts from burning during irradiation.

In use, with parts 4 and 6 or 80 in position and inert gas being supplied appropriately (which may be pre-heated to reduce any cooling effect that cool/cold gas might create), motor 104 causes holder 108 and in turn part 6 or 80 to rotate. Then, part 4 is urged by spring 118 towards part 6 or 80 with a constant force, whilst the V-shaped band defined between the parts is irradiated by the laser at a position at the bottom of the V-shaped band. Welding takes place as described above to define generally the arrangement shown in FIG. 8. During welding, a Prolas (Trade Mark) Weld Control weld temperature monitor may be used to control laser power output.

The parts 4, 6, 80 may be machined from injection moulded or extruded blanks. The parts may consist essentially of PEEK-OPTIMA (Trade Mark) or may comprise a major amount of PEEK-OPTIMA (Trade Mark) in combination with an absorber which is arranged to absorb radiation from the laser and facilitate the heating of the PEEK-OPTIMA polymer.

Where the polymer includes an absorber, the absorber may comprise a carbon black at 2 wt % (the balance being polymer). When a part includes an absorber, the part may be machined from a blank injection moulded using pellets comprising polymer and absorber.

Prior to laser welding, parts 4, 6, 80 are cleaned with methylated spirits to remove any traces of machining oil and dried overnight in an oven at 150° C. After removal from a drying oven the parts are welded within 20 minutes.

Table 1 provides details of parts 4, 6, 80 laser welded together and the conditions adopted.

| | | | Composition | | | | Laser spot | Weld temp during | Speed of |
|---|---|---|---|---|---|---|---|---|---|
| | Composition | Type of other | of part 6 | Ramp rate | Hold time | Power of | size | hold time | motor |
| Example No | of part 4 | part used | or 80 used | second | second | laser (W) | (mm) | (° C.) | rpm |
| 1 | PEEK-OPTIMA | Part 6 | PEEK-OPTIMA | 2 | 8 | 30-60 W | 3.0 | 425 | 22000 |
| | filled with | | filled with | | | | | | |
| | carbon black. | | carbon black. | | | | | | |
| 2 | PEEK-OPTIMA | Part 6 | Unfilled | 2 | 8 | | 3.0 | 425 | 22000 |
| | filled with | | PEEK-OPTIMA | | | | | | |
| | carbon black. | | | | | | | | |
| 3 | PEEK-OPTIMA | Part 80 | Unfilled | | 10 | 50 | 2.0 | — | 22000 |
| | filled with | | PEEK-OPTIMA | | | | | | |
| | carbon black. | | | | | | | | |
| 4 | PEEK-OPTIMA | Part 80 | Unfilled | | 20 | 35 | 2.5 | — | 22000 |
| | filled with | | PEEK-OPTIMA | | | | | | |
| | carbon black. | | | | | | | | |

Housings produced as described in Examples 1 to 4 were found to have strong welds between the housing parts which did not leak.

Housings as described may be implanted in a human body. They may contain any device, for example electronic device, which it is desired to implant. The device may be arranged to apply a stimulus to the body once implanted. It may be arranged to stimulate a nerve or muscle. Thus, said housing suitably contains electronic circuitry in a sealed environment.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of joining a first part to a second part, wherein the first part includes a first interface which is arranged to be joined to a second interface defined by the second part, wherein the first interface of the first part includes a first side and a second side, the method comprising:
directing radiation from a first position which is closer to the first side of the first interface to a position which is closer to the second side of the first interface, the radiation being directed to heat predominantly an area of the first interface of the first part which is closer to the second side of the first interface than it is to the first side of the first interface.

2. A method according to claim 1, wherein radiation is supplied from a radiation device which is arranged to heat a said area of the first interface.

3. A method according to claim 1, wherein said first interface defines a frusto-conical surface.

4. A method according to claim 1, wherein the thickness of the first part underlying said first interface and measured in a direction which is perpendicular to the first interface is smaller in a region which underlies said first interface at a position which is closer to the second side of the first interface than it is to said first side.

5. A method according to claim 1, wherein said first part tapers inwardly on moving from its first side to its second side.

6. A method according to claim 1, wherein said first part defines a pointed region at its second side.

7. A method according to claim 1, wherein said first part includes a first guide surface which is arranged to guide movement of the second part as the first and second parts are engaged prior to being joined, wherein said first guide surface faces outwardly and a first gap is defined between said first guide surface and an inwardly facing surface of said first part; wherein said second part includes a second guide surface which is arranged to cooperate with the first part as the first and second parts are engaged prior to being joined, said second guide surface facing inwardly and a second gap being defined between said second inwardly facing surface of said second part and a first guide surface of said first part.

8. A method according to claim 7, wherein said respective first and second gaps are defined by said first and second parts and the gaps are aligned to define a combined gap, wherein the combined gap is arranged to accommodate elements of said first and second interface regions after the first and second parts have been joined in the method.

9. A method according to claim 1, wherein the first and second parts are selected and engaged so that the first and second interfaces face one another and a region of the first interface abuts a region of the second interface.

10. A method according to claim 1, wherein the first and second interfaces together define a groove which is annular and outwardly facing and radiation is directed towards the bottom or closed end of the groove, in the method.

11. A method according to claim 1, which comprises causing the first and second parts to move relative to one another.

12. A method according to claim 1, wherein the first interface is softened so that it turns towards the second interface and makes face to face contact therewith in the method.

13. A method according to claim 1, wherein the method comprises causing the first and second parts to rotate so that radiation is directed around respective annular parts of the first and second parts being joined together.

14. A method according to claim 1, wherein said first part and/or said second part comprise a plastics material which comprises a biocompatible polymeric material.

15. A method according to claim 1, wherein said first and second parts comprise a biocompatible polymeric material which consists essentially of a repeat unit of formula (XX) 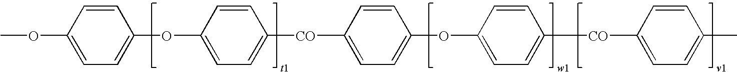
where t1 and w1 independently represent 0 or 1 and v1 represents 0, 1 or 2.

16. A method according to claim 1, wherein said first and/or second parts comprise a plastic material which is polyetheretherketone.

17. A method according to claim 1, wherein said first part includes radiation absorbing means which is arranged to absorb applied radiation and thereby allow said area of said first interface to be heated.

18. A method according to claim 1, wherein said first and second parts are arranged to be joined to define a housing suitable for implantation in a human body.

19. A housing made in the method according to claim 1.

20. A housing, wherein said first part includes a first guide surface which faces outwardly and abuts a second guide surface which faces inwardly wherein a gap is defined outwardly of said first guide surface in which joined interfaces of the first and second parts are contained, at least in part.
